(19) United States
(12) Patent Application Publication
(10) Pub. No.: US 2023/0149155 A1
(43) Pub. Date: May 18, 2023

(54) SURGICAL IMPLANT DELIVERY WITH DAMPING

(71) Applicant: Alcon Inc., Fribourg (CH)

(72) Inventors: Harlen Hoang, Fort Worth, TX (US); Yinghui Hu, Cedar Hill, TX (US); R. Mitchell Sherry, Fort Worth, TX (US); Douglas Brent Wensrich, Bedford, TX (US); Tuoqi Li, Irving, TX (US)

(21) Appl. No.: 18/048,580

(22) Filed: Oct. 21, 2022

Related U.S. Application Data
(60) Provisional application No. 63/263,948, filed on Nov. 12, 2021.

(51) Int. Cl. A61F 2/16

(52) U.S. Cl. CPC A61F 2/167; A61F 2/1678

(57) ABSTRACT

An apparatus for eye surgery may comprise a nozzle having a delivery lumen, an implant bay coupled to the nozzle, an implant disposed in the implant bay, and an actuator coupled to the implant bay. The actuator may comprise a housing; a bore through the housing; a plunger disposed within the bore; and a compression ring. The plunger can be configured to advance within the bore, thereby advancing the implant from the implant bay through the delivery lumen, and the bore can be configured to compress the compression ring as the plunger moves through the bore. In some embodiments, the bore can be configured to increase compression on the compression ring as the first end of the plunger moves through the bore.

### PRIORITY CLAIM

This application claims the benefit of priority of U.S. Provisional Pat. Application Serial No. 63/263,948 titled “SURGICAL IMPLANT DELIVERY WITH DAMPING,” filed on Nov. 12, 2021, whose inventors are Harlen Hoang, Yinghui Wu, R. Mitchell Sherry, Douglas Brent Wensrich and Tuoqi Li, which is hereby incorporated by reference in its entirety as though fully and completely set forth herein.

### TECHNICAL FIELD

The invention set forth in the appended claims relates generally to eye surgery. More particularly, but without limitation, the claimed subject matter relates to systems, apparatuses, and methods for inserting an implant into an eye.

### BACKGROUND

The human eye can suffer a number of maladies, which can cause mild deterioration to complete loss of vision. While contact lenses and eyeglasses can compensate for some ailments, ophthalmic surgery may be required for others. In some instances, implants may be beneficial or desirable. For example, an intraocular lens may replace a clouded natural lens within an eye to improve vision.

While the benefits of intraocular lenses and other implants are known, improvements to delivery systems, components, and processes continue to improve outcomes and benefit patients.

### BRIEF SUMMARY

New and useful systems, apparatuses, and methods for eye surgery are set forth in the appended claims. Illustrative embodiments are also provided to enable a person skilled in the art to make and use the claimed subject matter.

For example, some embodiments may comprise or consist essentially of an apparatus for delivering an implant, such as an intraocular lens. Such a delivery apparatus can be used to fold and compress an implant and deliver it into the eye through a narrow nozzle tip inserted into a small incision in various locations and using various surgical techniques. Some embodiments can be operated manually, using a single hand to advance the implant with a plunger or push rod. In some embodiments, the apparatus may comprise or consist of a means for smoothly, consistently, and predictably advancing an implant from an initial position to a dwell position, then from the dwell position into the eye. For example, an elastomeric ring can smoothen the implant advancement, dampen axial delivery resistance changes, and increase the consistency and controllability of implant delivery. In some examples, the apparatus may have a plunger disposed within a bore, and the ring can be compressed in the bore at different locations and/or at different rates to generate the right damping force at the right time or location during implant delivery. Additionally, the ring damping mechanism can also improve the smoothness of advancement of the implant from its initial position to a dwell position.

More generally, some embodiments of an apparatus for eye surgery may comprise a nozzle having a delivery lumen, an implant bay coupled to the nozzle, an implant disposed in the implant bay, and an actuator coupled to the implant bay. The actuator may comprise a housing, the housing having a first end proximate to the implant bay and a second end distal to the implant bay; a bore through the housing; a plunger having a first end and a second end, the first end disposed within the bore; and a compression ring disposed around the plunger within the bore between the plunger and the housing. The compression ring can be disposed between the first end and the second end of the plunger. The first end of the plunger can be configured to advance within the bore toward the first end of the housing, thereby advancing the implant from the implant bay through the delivery lumen, and the bore can be configured to compress the compression ring as the first end of the plunger moves through the bore. In some embodiments, the bore can be configured to increase compression on the compression ring as the first end of the plunger moves through the bore.

In more particular embodiments, at least a portion of the bore is tapered adjacent to the first end of the housing. For example, the bore may have a first width adjacent to the first end of the housing, the bore may have a second width adjacent to the second end of the housing, and the first width is less than the second width. In yet more particular embodiments, the bore may comprise a first region adjacent to the first end of the housing and a second region adjacent to the second end of the housing, the first region can have a width that decreases from the second width adjacent to the second region to the first width adjacent to the first end of the housing, and the second region can have a width that is substantially constant and equal to the second width.

The compression ring may comprise or consist essentially of an elastomer. For example, some embodiments of the compression ring may comprise or consist essentially of silicone, perfluoroelastomer (FFKM), nitrile rubber, fluorocarbon type A, chloroprene, polyurethane, or polytetrafluoroethylene. The housing may comprise or consist essentially of substantially rigid material, such as polypropylene (PP), polycarbonate (PC), acrylonitrile-butadlene-styrene (ABS), or polyoxymethylene (POM).

Some embodiments of an apparatus for delivering an implant to an eye may comprise a housing configured to be coupled to an implant bay, the housing having a first end and a second end; a bore passing longitudinally through the housing from the first end to the second end, and a tapered portion adjacent to the first end; a plunger having a first end and a second end, wherein the first end is disposed within the bore; and a compression ring coupled to the plunger within the bore between the plunger and the housing. The first end of the plunger can be configured to advance within the bore toward the first end of the housing, and the tapered portion can be configured to compress the compression ring as the first end of the plunger moves through the bore.

Some embodiments of an apparatus for eye surgery may comprise a nozzle having a delivery lumen, an implant bay coupled to the nozzle, an implant disposed in the implant bay, and an actuator coupled to the implant bay. The actuator may comprise a housing consisting essentially of polypropylene, the housing having a first end proximate to the implant bay and a second end distal to the implant bay; a bore through the housing, the bore having a tapered portion adjacent to the first end of the housing, and a fixed width between the tapered portion and the second end, the tapered portion reducing a width of the bore from the second end of the housing to the first end of the housing; a plunger having a first end and a second end, the first end disposed within the bore; an implant interface coupled to the first end of the plunger and configured to engage the implant; and a compression ring coupled to the plunger adjacent to the first end, the compression ring consisting essentially of silicone. The first end of the plunger can be configured to advance within the bore toward the first end of the housing, thereby advancing the implant from the implant bay through the delivery lumen, and the tapered portion of the bore can be configured to compress the compression ring as the first end of the plunger moves through the tapered portion.

Features, elements, and aspects described in the context of some embodiments may also be omitted, combined, or replaced by alternative features. Other features, objectives, advantages, and a preferred mode of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and a preferred mode of making and using some embodiments of the claimed subject matter. Like reference numbers represent like parts in the examples.

FIG. 1 is an auxiliary view of an example apparatus for delivering an implant into an eye.

FIG. 2 is a section view of the apparatus of FIG. 1.

FIG. 3A, FIG. 3B, and FIG. 3C are schematic diagrams illustrating additional details that may be associated with operation of the apparatus of FIG. 1.

FIG. 4 is a chart illustrating an example of a delivery force profile that may be associated with some embodiments of the apparatus of FIG. 1.

FIG. 5A and FIG. 5B are schematic diagrams illustrating an example use of the apparatus of FIG. 1 to deliver an implant to an eye.

### DESCRIPTION OF EXAMPLE EMBODIMENTS

The following description of example embodiments provides information that enables a person skilled in the art to make and use the subject matter set forth in the appended claims, but it may omit certain details already well known in the art. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position to receive an implant. However, as should be recognized by those skilled in the art, this frame of reference is merely a descriptive expedient rather than a strict prescription.

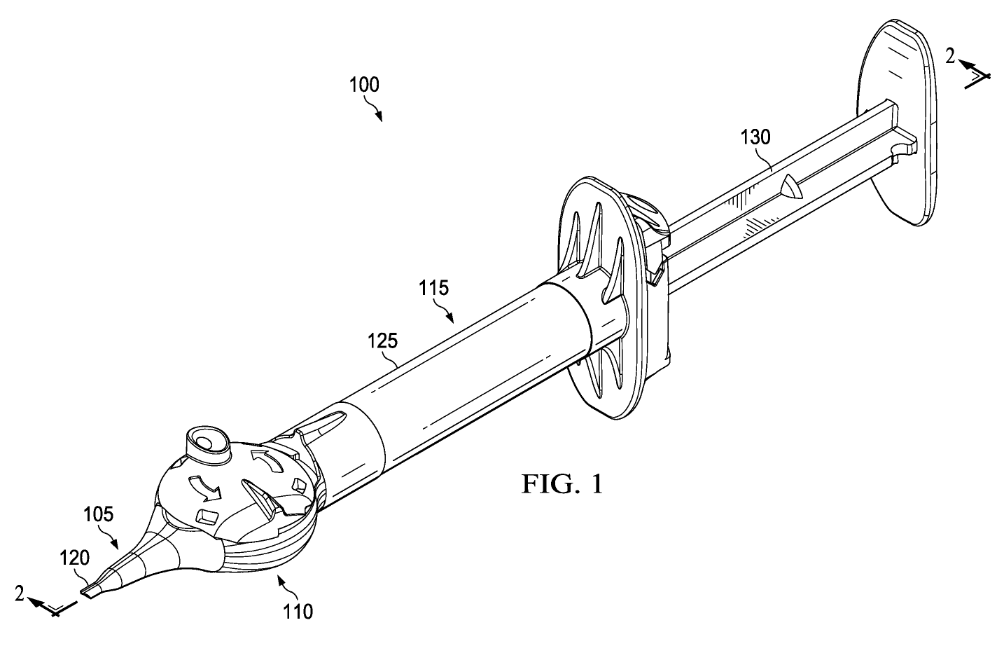

FIG. 1 is an auxiliary view of an example of an apparatus 100 that can deliver an implant into an eye. In some embodiments, the apparatus 100 may comprise two or more modules, which can be configured to be coupled and decoupled as appropriate for storage, assembly, use, and disposal. As illustrated in FIG. 1, some embodiments of the apparatus 100 may include a nozzle 105, an implant bay 110 coupled to the nozzle 105, and an actuator 115 coupled to the implant bay 110.

The nozzle 105 generally comprises a tip 120 adapted for insertion through an incision into an eye. The size of the tip 120 may be adapted to surgical requirements and techniques as needed. For example, small incisions are generally preferable to reduce or minimize healing times. Incisions of less than 2 millimeters may be preferable in some instances, and the tip 120 of the nozzle 105 may have a width of less than 2 millimeters in some embodiments. For example, in more particular embodiments, the tip 120 may have a width between about 1.5 millimeters and about 2 millimeters.

The implant bay 110 generally represents a wide variety of apparatuses that are suitable for storing an implant prior to delivery into an eye. In some embodiments, the implant bay 110 may additionally or alternatively be configured to prepare an implant for delivery. For example, some embodiments of the implant bay 110 may be configured to be actuated by a surgeon or other operator to prepare an implant for delivery by subsequent action of the actuator 115. In some instances, the implant bay 110 may be configured to actively deform, elongate, extend, or otherwise manipulate features of the implant before the implant is advanced into the nozzle 105. For example, the implant bay 110 may be configured to fold, tuck, extend or splay one or more features, such as haptics, of an intraocular lens.

The actuator 115 is generally configured to advance an implant from the implant bay 110 into the nozzle 105, and thereafter from the nozzle 105 through an incision and into an eye. The actuator 115 of FIG. 1 generally comprises a housing 125 and a plunger rod 130. The housing 125 may be comprised of or consist essentially of a substantially rigid polymer. For example, polypropylene or similar rigid plastics, such as polycarbonate (PC), acrylonitrile-butadlene-styrene (ABS), or polyoxymethylene (POM) may be suitable for some embodiments. In other embodiments, other medical-grade materials may also be suitable, such as stainless steel, aluminum, or titanium, or example. The plunger rod 130 is generally comprised of a substantially rigid material, such as a medical grade polymer material.

In general, components of the apparatus 100 may be coupled directly or indirectly. For example, the nozzle 105 may be directly coupled to the implant bay 110 and may be indirectly coupled to the actuator 115 through the implant bay 110. Coupling may include fluid, mechanical, thermal, electrical, or chemical coupling (such as a chemical bond), or some combination of coupling in some contexts. For example, the implant bay 110 may be mechanically coupled to the actuator 115 and may be mechanically and fluidly coupled to the nozzle 105. In some embodiments, components may also be coupled by virtue of physical proximity, being integral to a single structure, or being formed from the same piece of material.

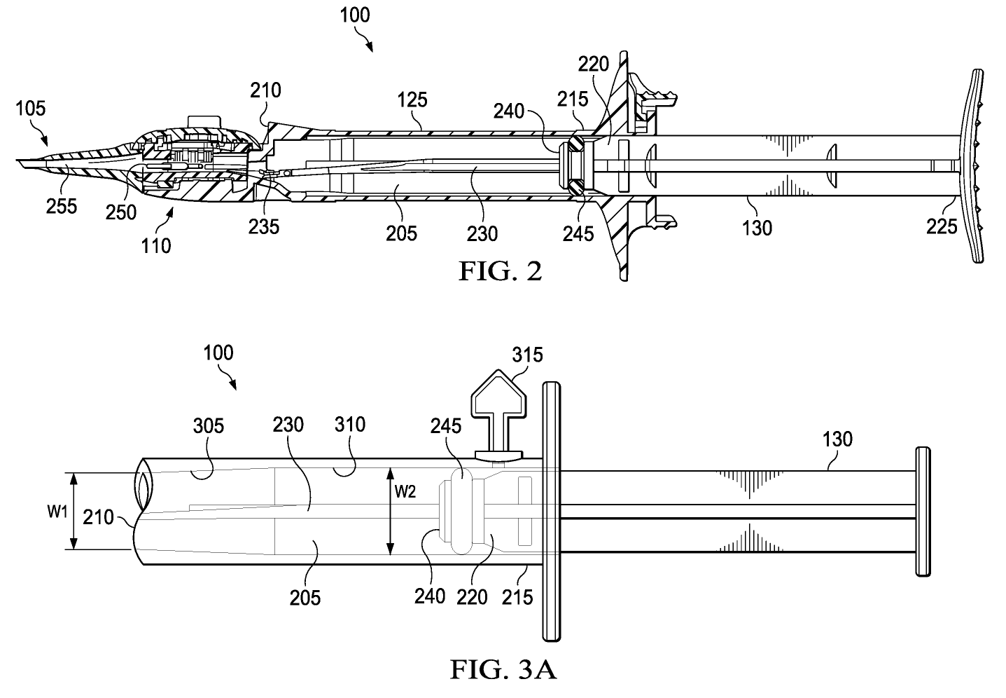

FIG. 2 is a section view of the apparatus 100 of FIG. 1, illustrating additional details that may be associated with some embodiments. For example, a bore 205 generally passes through the housing 125 of FIG. 2, longitudinally from a first end 210 of the housing 125 to a second end 215 of the housing 125. In the example of FIG. 2, the first end 210 is disposed proximate to the implant bay 110, and the second end 215 is disposed distal to the implant bay 110.

The plunger rod 130 may be disposed at least partially within the bore 205. For example, as illustrated in FIG. 2, the plunger rod 130 may have a first end 220 disposed within the bore 205. A second end 225 may extend from the bore 205. A plunger 230 may also be disposed within the bore 205. As shown in the example of FIG. 2, some embodiments of the plunger 230 may have a tip 235 and a head 240. The head 240 may be coupled to the first end 220 of the plunger rod 130.

A compression ring 245 may be disposed within the bore 205 between the housing 125 and the plunger rod 130 or the plunger 230. In some embodiments, the compression ring 245 can be disposed around the plunger 230 as illustrated in the example of FIG. 2. In more particular examples, the compression ring 245 may be coupled to the head 240. The compression ring 245 is representative of a ring, collar, sleeve, or similar rounded profile. While the compression ring 245 of FIG. 2 is round, the compression ring 245 may have other shapes. In general, the compression ring 245 may have any shape configured to provide contact between the plunger rod 130 or the plunger 230 and the bore 205. In some embodiments, the compression ring 245 may be continuous, as illustrated in the example of FIG. 2; in other embodiments, the compression ring 245 may consist of discrete contact points around the plunger rod 130 or the plunger 230. For example, a rectangular or cross shape may be suitable for some embodiments. In some embodiments, the compression ring 245 may comprise or consist essentially of silicone, perfluoroelastomer (FFKM), nitrile rubber, fluorocarbon type A, chloroprene, polyurethane, or polytetrafluoroethylene.

An implant 250 may be stored initially in the implant bay 110. In some embodiments, the implant bay 110 may additionally or alternatively be configured to prepare the implant 250 for delivery. For example, some embodiments of the implant bay 110 may be configured to be actuated by a surgeon or other operator to prepare the implant 250 for delivery by subsequent action of the actuator 115. In some instances, the implant bay 110 may be configured to actively deform, elongate, extend, or otherwise manipulate features of the implant 250 before the implant 250 is advanced into a delivery lumen 255 of the nozzle 105. For example, some embodiments of the implant bay 110 may be configured to orient or fold the implant 250. Some embodiments of the implant 250 may comprise one or more haptics, which can be oriented for delivery.

In use, the plunger rod 130 may be advanced within the bore 205 toward the first end 210 of the housing 125, thereby advancing the plunger 230, which can advance the implant 250 from the implant bay 110 through the delivery lumen 255. In the example of FIG. 2, the compression ring 245 can be configured to advance with the plunger 230. More particularly, the compression ring 245 can be coupled to the head 240, between the housing 125 and the plunger 230. The material properties of the housing 125 and the compression ring 245 can reduce static friction between them, allowing the compression ring 245 to slide smoothly against the inner wall of the housing 125 as the plunger 230 is advanced. The housing 125 may be configured to compress the compression ring 245 as it is advanced. For example, at least a portion of the bore 205 may be tapered adjacent to the first end 210 of the housing 125 to compress the compression ring 245.

In the example of FIG. 2, the tip 235 of the plunger 230 can be configured to contact or otherwise engage the implant 250 to advance the implant 250 from the implant bay 110 through the nozzle 105 as the plunger 230 is advanced. At least a portion of the plunger 230 may extend into or through the implant bay 110 and/or the nozzle 105. As the plunger 230 is fully advanced, the tip 235 can eject the implant 250 from the nozzle 105.

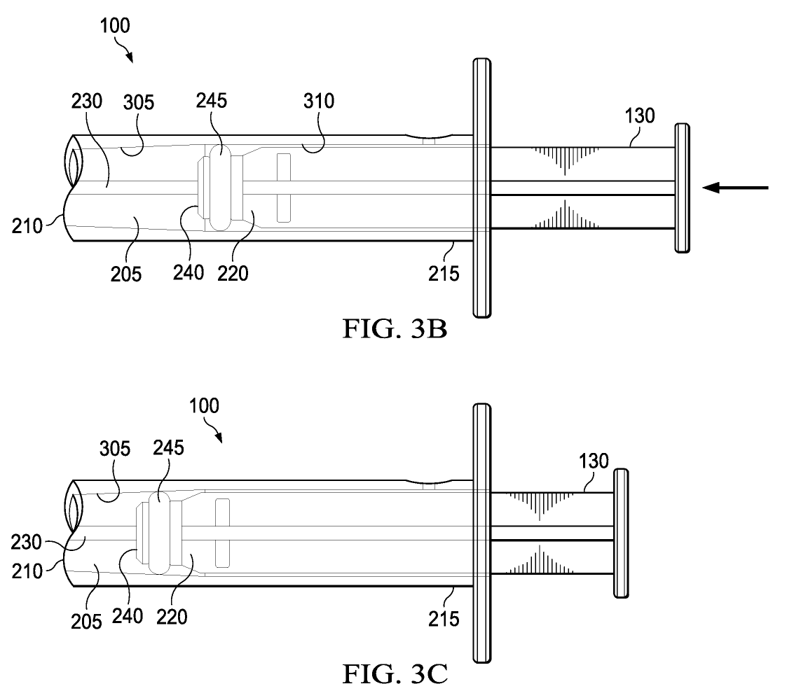

FIG. 3A, FIG. 3B, and FIG. 3C are schematic diagrams illustrating additional details that may be associated with operation of the apparatus 100 as the plunger 230 moves from a first position through a second position to a third position. FIG. 3A illustrates a portion of an example of the apparatus 100 in a first state, FIG. 3B illustrates the apparatus 100 of FIG. 3A in a second state, and FIG. 3C illustrates the apparatus 100 of FIG. 3A in a third state.

In each of the three states of FIG. 3A, FIG. 3B, and FIG. 3C, at least a portion of the bore 205 is tapered adjacent to the first end 210 of the housing 125. The tapered portion can reduce the width of the bore 205 between the second end 215 and the first end 210. For example, the bore 205 of FIG. 3A may be cylindrical, and the tapered portion may reduce the diameter of the bore 205. In the illustrated examples, the bore 205 has a first width W1 adjacent to the first end 210, a second width W2 adjacent to the second end 215, and the first width W1 is less than the second width W2. In some embodiments, the bore 205 may comprise a first region 305 adjacent to the first end 210 and a second region 310 adjacent to the second end 215. As illustrated in the example of FIG. 3A, the first region 305 may be a tapered portion that reduces the width of the bore 205 from the second width W2 to the first width W1 adjacent to the first end 210. The second region 310 may have a width that is substantially constant or fixed and equal to the second width W2.

In the first state of the apparatus 100 illustrated in FIG. 3A, the plunger 230 may be retained in the first position by a plunger lock 315, which may be suitable for maintaining the apparatus 100 in the first state for shipping, storage, and surgical preparation, for example. In the first position, the compression ring 245 may be under low compression or no compression. The plunger lock 315 may be removed to allow the plunger 230 to be advanced within the bore 205.

In the second state of the apparatus 100 illustrated in FIG. 3B, the plunger lock 315 is removed and the plunger 230 is advanced to the second position. As the plunger 230 is advanced from the first position of FIG. 3A to the second position of FIG. 3B, the constant width of the second region 310 maintains a consistent compression force on the compression ring 245, which can provide a smooth and controlled feel to an operator.

As the plunger 230 is advanced further through the bore 205, a tapered portion can compress the compression ring 245. As illustrated in FIG. 3C, for example, the compression ring 245 may be compressed as it moves into and through the first region 305. In more particular embodiments, a tapered portion may gradually increase the compression on the compression ring 245 as it is advanced closer toward the implant bay 110 (not shown in FIG. 3C).

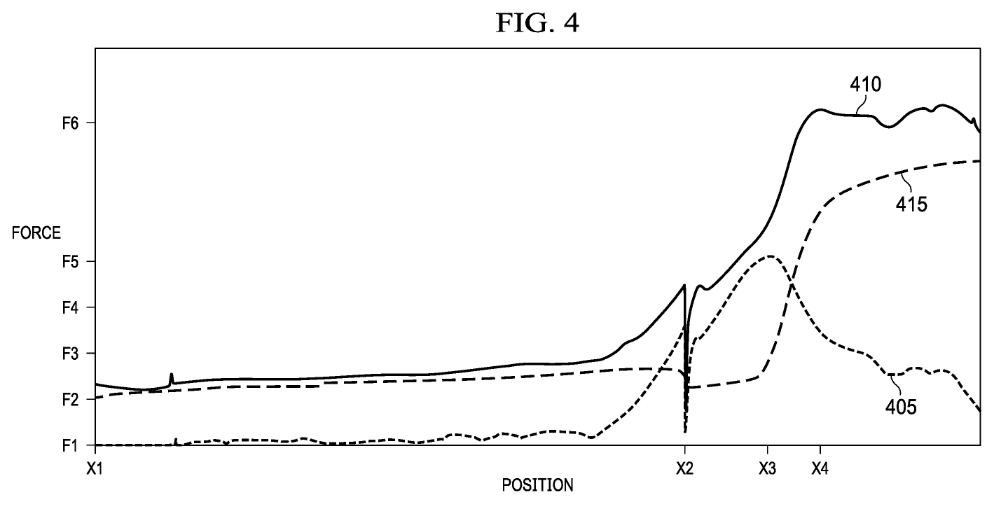

FIG. 4 is a chart illustrating an example of a delivery force profile that may be associated with some embodiments of the apparatus 100. FIG. 4 also illustrates an example of a delivery force profile that may be associated with other apparatuses. The horizontal axis represents the relative position of a portion of the plunger, such as the tip 235 or the head 240, as it advances through a bore, such as the bore 205. The vertical axis represents the forces on the apparatus. For example, Line 405 illustrates a delivery force that may be associated with such an apparatus not having the compression ring 245 or a tapered portion within the bore 205. Line 410 illustrates an example of the delivery force of the apparatus 100 as the plunger 230 is advanced with the compression ring 245. In general, the delivery force is equal to the minimal amount of force that must be applied by a surgeon or other operator to advance a plunger and deliver an implant. For example, in the context of FIGS. 3A-3C, the delivery force is equal to the minimal force that must be applied to the plunger rod 130 to overcome the resistance between the compression ring 245 and the bore 205. Line 415 illustrates the resistance force provided by the compression ring 245 as it advances through the bore 205.

As FIG. 4 illustrates, the delivery force of Line 405 remains relatively constant around F1, and the delivery force of Line 410 and the resistance force of Line 415 remain relatively constant around F2, as the plunger advances from the initial position X1, such as illustrated in FIG. 3A, toward the position X2. The delivery forces of Line 405 and Line 410 increase slightly to about F3 and F4, respectively, prior to the position X2.

At position X2, the implant has generally been advanced from the implant bay into the delivery lumen and advancement of the plunger is paused, which can allow the implant to be inspected for proper orientation before delivery. Consequently, the delivery force represented in each of Line 405 and Line 410 drops as the result allowing the implant to dwell briefly in this period. The position of the compression ring at position X2 is generally represented in FIG. 3B, in which the compression ring 245 is disposed at the juncture between the first region 305 and the second region 310.

As the plunger is advanced beyond the position X2, the delivery forces continue to increase as illustrated by Line 405 and Line 410, which is generally the result of the implant entering the delivery lumen (see, e.g., delivery lumen 255 of FIG. 2). In the example of Line 405, without the compression ring 245 or a taper in the bore 205, the delivery force peaks at about F5 around point X3 before drastically dropping. The sudden drop is generally the result of the implant passing through the tip (e.g., tip 120 of FIG. 1), and more particularly, the result of the maximum compression of the implant passing through the tip. In the example of Line 410, the delivery force continues to increase smoothly as resistance of the compression ring 245 increases, as illustrated in Line 415. For example, as illustrated in FIG. 3C, the compression ring 245 advances into the first region 305, which is tapered to increase the forces on the compression ring 245 as the implant passes through the tip. The delivery force then plateaus around position X4 to provide a relatively constant delivery force even as the implant passes through the tip. In the example of FIG. 4, the delivery force plateaus at about F6. In some embodiments, F6 may be substantially similar to F4 or F5.

Thus, as FIG. 4 illustrates, the plunger may be advanced from a first position to a second position with a relatively constant first delivery force, from the second position to a third position with a second delivery force, and from the third position to a fourth position with a relatively constant third delivery force, wherein the third delivery force is greater than the first delivery force. More generally, the compression ring 245 can provide a damping effect on the delivery forces as the implant is advanced and ejected through the tip. Additionally, the position of the compression ring 245 relative to the plunger tip, the taper of the bore 205, or both can be adjusted to optimize the timing of the damping. For example, the compression ring 245 can be moved forward to advance the damping or can be moved aft to delay the damping.

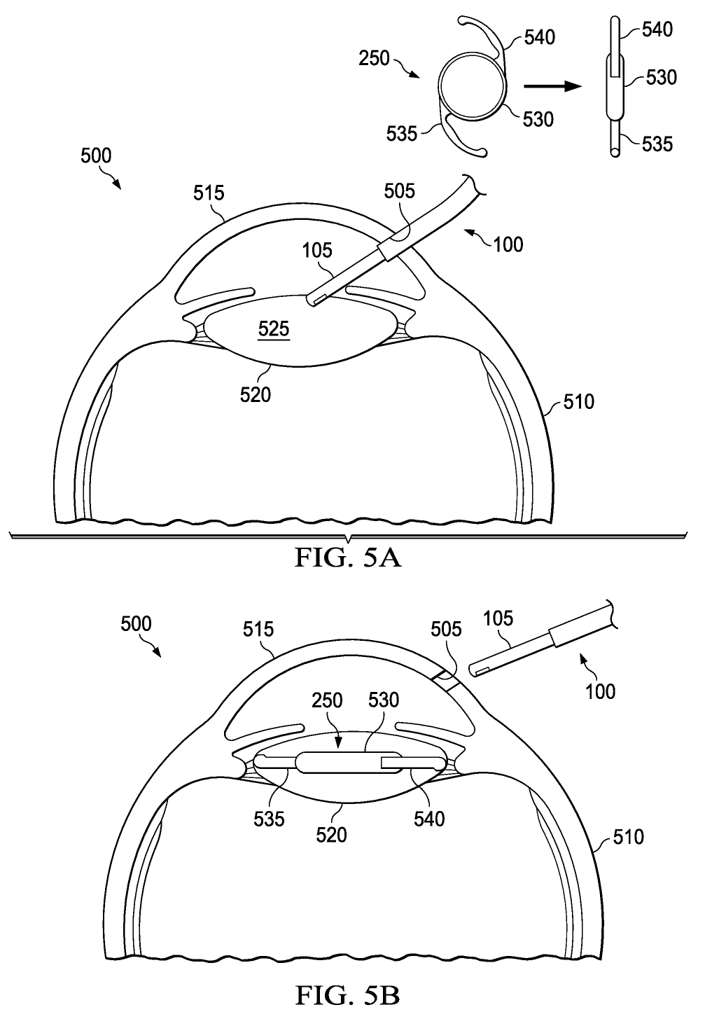

FIG. 5A and FIG. 5B are schematic diagrams illustrating an example use of the apparatus 100 of FIG. 1 to deliver the implant 250 to an eye 500. As illustrated, an incision 505 may be made in the eye 500 by a surgeon, for example. In some instances, the incision 505 may be made through the sclera 510 of the eye 500. In other instances, an incision may be formed in the cornea 515 of the eye 500. The incision 505 may be sized to permit insertion of a portion of the nozzle 105 to deliver the implant 250 into the capsular bag 520. For example, in some instances, the size of the incision 505 may have a length less than about 3000 microns (3 millimeters). In other instances, the incision 505 may have a length of from about 1000 microns to about 1500 microns, from about 1500 microns to about 2000 microns, from about 2000 microns to about 2500 microns, or from about 2500 microns to about 3000 microns.

After the incision 505 is made, the nozzle 105 can be inserted through the incision 505 so that the width of the tip 120 aligns with the length of the incision 505, allowing the nozzle 105 to extend into an interior portion 525 of the eye 500. The apparatus 100 can then eject the implant 250 through the nozzle 105 into the capsular bag 520 of the eye 500, substantially as described with reference to FIG. 2.

In some embodiments, the implant 250 may comprise an intraocular lens having a shape similar to that of a natural lens of an eye, and it may be made from numerous materials. Examples of suitable materials may include silicone, acrylic, and combinations of such suitable materials. In some instances, the implant 250 may comprise an intraocular lens that is fluid-filled, such as a fluid-filled accommodating intraocular lens. The implant 250 may also comprise an intraocular lens that includes one or more features, such as haptics, for positioning the intraocular lens within an eye. In the example of FIG. 5A and FIG. 5B, the implant 250 is illustrative of an intraocular lens having an optic body 530, a leading haptic 535, and a trailing haptic 540.

The implant 250 may be delivered in a folded configuration and can revert to a resting state with the leading haptic 535 and the trailing haptic 540 being at least partially curved around the optic body 530, within the capsular bag 520, as shown in FIG. 5B. The capsular bag 520 can retain the implant 250 within the capsular bag 520 in a relationship relative to the eye 500 so that the optic body 530 refracts light directed to the retina (not shown). The leading haptic 535 and the trailing haptic 540 can engage the capsular bag 520 to secure the implant 250 therein. After delivering the implant 250 into the capsular bag 520, the nozzle 105 may be removed from the eye 500 through the incision 505, and the eye 500 can be allowed to heal over time.

The systems, apparatuses, and methods described herein may provide significant advantages. Some embodiments may be particularly advantageous for improving the delivery of intraocular lenses, making it smoother, more consistent, and more predictable throughout the delivery procedure. For example, the compression ring 245 and the bore 205 may be configured to dampen delivery forces as a lens or other implant is delivered. In more particular examples, the compression ring 245 can be compressed in the bore at different locations and/or at different rates to generate the right damping force at the right time and/or location to substantially reduce the risk of sudden movement throughout the procedure. Significantly, the position of the compression ring 245 can be modified for different embodiments to optimize the timing and location of the damping force as desired.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations, the nozzle 105, the implant bay 110, and the actuator 115 may each be separated from one another or combined in various ways for manufacture or sale.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. An apparatus for eye surgery, the apparatus comprising:
a nozzle having a delivery lumen;
an implant bay coupled to the nozzle;
an implant disposed in the implant bay; and
an actuator coupled to the implant bay, the actuator comprising:
a housing having a first end proximate to the implant bay and a second end distal to the implant bay,
a bore through the housing from the first end of the housing to the second end of the housing,,
a plunger having a first end and a second end, the first end disposed within the bore, and
a compression ring disposed around the plunger within the bore between the plunger and the housing;
wherein the first end of the plunger is configured to advance within the bore toward the first end of the housing, thereby advancing the implant from the implant bay through the delivery lumen, and the bore is configured to compress the compression ring as the first end of the plunger moves through the bore.

2. The apparatus of claim 1, wherein at least a portion of the bore is tapered adjacent to the first end of the housing.

3. The apparatus of claim 1, wherein:
the bore has a first width adjacent to the first end of the housing;
the bore has a second width adjacent to the second end of the housing; and
the first width is less than the second width.

4. The apparatus of claim 1, wherein:
the bore has a first width adjacent to the first end of the housing;
the bore has a second width adjacent to the second end of the housing;
the bore comprises a first region and a second region;
the first region has a width that decreases from the second width adjacent to the second region to the first width adjacent to the first end of the housing; and
the second region has a width that is substantially constant and equal to the second width.

5. The apparatus of claim 1, wherein the compression ring is disposed between the first end and the second end of the plunger.

6. The apparatus of claim 1, wherein the bore is configured to increase compression on the compression ring as the first end of the plunger moves through the bore.

7. The apparatus of claim 1, wherein the plunger comprises a tip configured to engage the implant.

8. The apparatus of claim 7, wherein the tip extends into the implant bay.

9. The apparatus of claim 1, wherein the compression ring comprises or consists essentially of an elastomeric material.

10. The apparatus of claim 1, wherein the compression ring comprises or consists essentially of silicone, perfluoroelastomer (FFKM), nitrile rubber, fluorocarbon type A, chloroprene, polyurethane, or polytetrafluoroethylene.

11. The apparatus of claim 1, wherein the housing comprises or consists essentially of polypropylene, polycarbonate, acrylonitrile-butadlene-styrene, or polyoxymethylene.

12. The apparatus of claim 1, wherein the housing comprises polypropylene and the compression ring comprises silicone.

13. An apparatus for delivering an implant to an eye, the apparatus comprising:
a housing configured to be coupled to an implant bay, the housing having a first end and a second end;
a bore passing longitudinally through the housing from the first end of the housing to the second end of the housing and having a tapered portion adjacent to the first end of the housing;
a plunger having a first end and a second end, wherein the first end is disposed within the bore; and
a compression ring coupled to the plunger within the bore between the plunger and the housing;
wherein the first end of the plunger is configured to advance within the bore toward the first end of the housing, and the tapered portion is configured to compress the compression ring as the first end of the plunger moves through the bore.

14. The apparatus of claim 13, wherein the tapered portion reduces a width of the bore from the second end of the housing to the first end of the housing.

15. The apparatus of claim 13, wherein:
the bore has a first width adjacent to the first end of the housing;
the bore has a second width adjacent to the second end of the housing; and
the first width is less than the second width.

16. The apparatus of claim 13, wherein the bore has a fixed width between the tapered portion and the second end of the housing.

17. The apparatus of claim 13, wherein the plunger comprises a tip configured to engage the implant.

18. The apparatus of claim 17, wherein the tip extends into the implant bay.

19. The apparatus of claim 13, wherein the compression ring comprises or consists essentially of silicone, perfluoroelastomer (FFKM), nitrile rubber, fluorocarbon type A, chloroprene, polyurethane, or polytetrafluoroethylene.

20. The apparatus of claim 13, wherein the housing comprises or consists essentially of polypropylene or polyoxymethylene.

21. The apparatus of claim 13, wherein the housing comprises polypropylene and the compression ring comprises silicone.

22. An apparatus for eye surgery, the apparatus comprising:
a nozzle having a delivery lumen;
an implant bay coupled to the nozzle;
an implant disposed in the implant bay; and
an actuator coupled to the implant bay, the actuator comprising:
a housing consisting essentially of polypropylene, the housing having a first end proximate to the implant bay and a second end distal to the implant bay,
a bore through the housing, the bore having a tapered portion adjacent to the first end of the housing, and a fixed width between the tapered portion and the second end of the housing, the tapered portion reducing a width of the bore from the second end of the housing to the first end of the housing,
a plunger disposed within the bore, the plunger having a tip and a head, and
a compression ring coupled to the plunger adjacent to the head, the compression ring consisting essentially of silicone;
wherein the first end of the plunger is configured to advance within the bore toward the first end of the housing, thereby advancing the implant from the implant bay through the delivery lumen, and the tapered portion of the bore is configured to compress the compression ring as the first end of the plunger moves through the tapered portion.

23. (canceled)
